(19) United States
(12) Patent Application Publication
(10) Pub. No.: US 2010/0131052 A1
(43) Pub. Date: May 27, 2010

(54) METHOD FOR PRODUCING A CORROSION-INHIBITING COATING ON AN IMPLANT MADE OF A BIOCORRODIBLE MAGNESIUM ALLOY AND IMPLANT PRODUCED ACCORDING TO THE METHOD

(76) Inventors: Gerhard Kappelt, Erlangen (DE); Peter Kurze, Nideggen (DE); Dora Banerjee, Kerpen (DE); Nina Adden, Nuernberg (DE)

Correspondence Address: GREER, BURNS & CRAIN, 300 S WACKER DR, 25TH FLOOR, CHICAGO, IL, 60606, US

(21) Appl. No.: 12/614,020

(22) Filed: Nov. 6, 2009

(30) Foreign Application Priority Data
Nov. 21, 2008 (DE) 10 2008 043 970.3

(51) Int. Cl. A61F 2/82

(52) U.S. Cl. 623/1.46

(57) ABSTRACT

The invention relates to a method for producing corrosion-inhibiting coatings on an implant made of a biocorrodible magnesium alloy and to implants obtained or obtainable according to the method.

The invention relates to two methods for producing corrosion-inhibiting coatings on an implant made of a biocorrodible magnesium alloy and to implants obtained or obtainable according to the method.

### TECHNOLOGICAL BACKGROUND AND STATE OF THE ART

Implants are employed in a wide variety of forms in modern medical technology. They are used, among other things, for the support of vessels, hollow organs and vein systems (endovascular implants), for the fastening and temporary fixation of tissue implants and tissue transplantations, but also for orthopedic purposes, such as a nail, plate or screw.

The implantation of stents is one of the most effective therapeutic measures for the treatment of vascular diseases. Stents have the purpose of assuming a supporting function in hollow organs of a patient. To this end, stents of conventional build have a filigree carrying structure made of metal struts, which is initially available in a compressed form for introduction into the body and is expanded at the site of the application. One of the main application areas of such stents is to permanently or temporarily widen and hold open vessel constrictions, particularly constrictions (stenosis) of coronary blood vessels. In addition, aneurysm stents are also known, which are used to support damaged vessel walls.

The base body of every implant, particularly of stents, is made of an implant material. An implant material is a non-living material, which is employed for applications in medicine and interacts with biological systems. A basic prerequisite for the use of a material as implant material, which is in contact with the body area when used as intended, is the body friendliness thereof (biocompatibility)). Biocompatibility shall be understood as the ability of a material to evoke an appropriate tissue response in a specific application. This includes an adaptation of the chemical, physical, biological, and morphological surface properties of an implant to the recipient's tissue with the aim of a clinically desirable interaction. The biocompatibility of the implant material is also dependent on the time process of the response of the biosystem in which it is implanted. For example, irritations and inflammations occur in a relatively short time, which can lead to tissue changes. As a function of the properties of the implant material, biological systems thus react in different ways. According to the response of the biosystem, the implant materials can be divided into bioactive, bioinert and degradable/resorbable materials. For the purpose of the present invention, only degradable/resorbable, metal implant materials are of interest, which below are referred to as biocorrodible metal materials.

Hence, the use of biocorrodible metal materials lends itself already simply because often the implant must only remain in the body temporarily in order to achieve the medical purpose. Implants made of permanent materials, which is to say materials that are not degraded in the body, optionally must be removed again, since the body may reject them in the medium and long-term even if biocompatibility is high.

One approach to avoid a further surgical operation is thus to form the entire implant, or parts thereof, from a biocorrodible metal material. Biocorrosion shall be understood as processes which are caused by the presence of body media and which result in a gradual degradation of the structure that is made of the material. At a certain point in time, the implant, or at least the part of the implant composed of the biocorrodible material, loses the mechanical integrity thereof. The degradation products are largely resorbed by the body. The degradation products, such as magnesium, in the best case have even a positive therapeutic effect on the surrounding tissue. Low quantities of non-resorbable alloying constituents—provided they are not toxic—can be tolerated.

Known biocorrodible metal materials include technical pure iron and biocorrodible alloys of the main elements of magnesium, iron, zinc, molybdenum and tungsten. In DE 197 31 021 A1 it is proposed, among other things, to form medical implants from a metal material, the main constituent thereof being an element from the group consisting of alkali metals, alkaline earth metals, iron, zinc and aluminum. Alloys based on magnesium, iron and zinc are described as being particularly suited. Minor constituents of the alloys can be manganese, cobalt, nickel, chromium, copper, cadmium, lead, tin, thorium, zirconium, silver, gold, palladium, platinum, silicon, calcium, lithium, aluminum, zinc and iron. For the purpose of the present invention, only biocorrodible magnesium alloys are of interest.

EP 1 419 793 B1 describes the use of a biocorrodible magnesium alloy having a fraction of magnesium >90 weight %, yttrium 3.7-5.5 weight %, rare earth metals 1.5-4.4 weight % and the remainder <1 weight % for production of a stent.

EP 1 842 507 A1 describes an implant comprising a base body, which is made of an yttrium-free and gadolinium-containing magnesium alloy. Furthermore, the alloy can include neodymium (Nd), zinc (Zn), zirconium (Zr) and calcium (Ca). The alloy preferably comprises 1.0 to 5.0 weight % Gd and 1.0 to 5.0 weight % Nd, in order to leave the cytotoxicity at a low level and improve mechanical properties, such as strength, hardness and ductility, in addition to the proccessability of the material. Contents of Zn as well as Zr preferably add up to 0.1 to 3.0 weight % in order to ensure a homogeneous distribution of the elements in the alloy.

Biodegradable vascular supports (stents) made of magnesium alloys have already been tested in clinical trials. For example, a magnesium alloy was used, which comprises yttrium and rare earths, the technical description being WE43. However, in the use of this alloy, which also has already been tested in other areas of implantology in animal experiments, some properties continue to cause problems in a physiological environment; these WE alloys in particular exhibit too fast a degradation in physiological media. The relatively fast biocorrosion of the magnesium alloys, particularly in the area of structures subject to high mechanical stresses, limits the use of the implants.

Both the fundamentals of magnesium corrosion and a large number of technical methods for improving the corrosion behavior (in terms of a strengthening of the corrosion protection) are known from the state of the art. For example, it is known that the addition of yttrium and/or further rare earth metals provides a magnesium alloy with slightly higher corrosion resistance in sea water.

Another approach provides for the creation of a corrosion-protecting layer on the shaped body made of the magnesium or a magnesium alloy. Known methods for creating a corrosion-protecting layer were developed and optimized with a view to a technical application of the shaped body—however not a medical application in biocorrodible implants in a physiological environment. These known methods comprise: applying polymers or inorganic cover layers, creating an enamel, chemically converting the surface, hot gas oxidation, anodizing, plasma sprayings, laser beam remelting, PVD methods, ion implantation or painting.

Conventional technical fields of application of shaped bodies made of magnesium alloys outside medical technology generally require that corrosive processes are largely suppressed. Accordingly, the goal of most technical methods is a complete inhibition of corrosive processes. In contrast, the goal for improving the corrosion behavior of biocorrodible magnesium alloys should not be the complete suppression, but only the inhibition of corrosive processes. Already for to this reason, most known methods are not suited for creating a corrosion protection coating. Furthermore, toxicological aspects must also be taken into consideration for a medical application. Moreover, corrosive processes are highly dependant on the medium in which they take place, and therefore, the findings for corrosion protection gained under conventional surrounding conditions in the field of technology cannot be transferred to the processes in a physiological environment without reservations. Finally, in a plurality of medical implants also the mechanisms underlying the corrosion process will likely deviate from common technical applications of the material. For example, stents, surgical suture material or clips are deformed mechanically while in use such that the sub-process of tension crack corrosion should be of significant importance during the degradation of these shaped bodies.

DE 101 63 106 A1 provides for a change of the magnesium material with respect to the corrosivity thereof by modification with halides. The magnesium material is to be used for the production of medical implants. A fluoride is preferably used as the halide. The modification of the material is achieved by adding salt-like halogen compounds by alloying. Accordingly, the composition of the magnesium alloy is changed by adding halides in order to reduce the corrosion rate. Thus, the entire shaped body, which is made of such a modified alloy, will have a modified corrosion behavior. Furthermore, additional material properties can be influenced by the addition by alloying, which are important for processing or which influence the mechanical properties of the shaped body produced with the material.

The object underlying the present invention is to provide alternative or preferably improved methods for producing a corrosion-inhibiting coating on an implant made of a biocorrodible magnesium alloy. The corrosion-inhibiting coating created by the method is to only cause temporary inhibition, but not complete suppression of the corrosion of the material in a physiological environment. In particular, an improved implant with respect to the corrosion behavior thereof, made of a biocorrodible magnesium alloy, is to be provided.

### SUMMARY OF THE INVENTION

The implant according to the invention achieves or reduces one or more of the above-described objects. The invention is based on an implant, which is entirely or partially made of a biocorrodible magnesium alloy.

This object is achieved according to a first variant by the claimed method for producing a corrosion-inhibiting coating on an implant made of a biocorrodible magnesium alloy. The first method comprises the following steps:
(i) Providing the implant made of a biocorrodible magnesium alloy; and
(ii) anodic plasma-chemical treatment of the implant surface in an aqueous, fluoride-free electrolyte, which comprises at least ammonia (NH₃), phosphoric acid (H₃PO₄) and boric acid (H₃BO₃).

An alternative, second method for producing a corrosion-inhibiting coating on an implant made of a biocorrodible magnesium alloy comprises the following steps.
(i) Providing the implant made of a biocorrodible magnesium alloy; and
(ii) anodic treatment of the implant surface with an aqueous electrolyte, which comprises at least sodium permanganate (NaMnO₄) and ammonium vanadate (NH₄VO₃).

It was shown that the creation of a coating in the above methods does not result in the formation of a protective layer that completely or largely inhibits corrosion in a physiological environment. In other words, in a physiological environment, corrosion of the implant still occurs, however significantly more slowly. The corrosion-inhibiting coating is produced in each case by a near-surface conversion of the material of the implant; thus no application of material onto a surface of the implant is carried out, instead a chemical conversion of the metal surface and of the different constituents of the electrolytes takes place.

Another significant and common advantage of the above-mentioned method is that the developing passivation layer is porous on the outside. As a result, the embedding of pharmaceutically active substances in pure form or appropriate galenics is possible, which is of high importance especially with respect to the use in implants, particularly stents.

Finally it is advantageous that the created passivation layers have high adhesion, but are not brittle and do not burst under mechanical stress. In fact, it was shown in preliminary studies using stents that significant mechanical stresses can be tolerated, without the passivation layer experiencing significant worsening or parts of the passivation layer becoming fragmented.

The first method variant using an anodic plasma-chemical treatment of the implant surface is based, in a modified way, on a technical passivation method that is known per se, which in literature is referred to as anodic oxidation by spark discharge (ANOF), spark discharges in electrolytes, anodic spark deposition (ASD), micro-arc oxidation, high-voltage anodizing, plasma-electrolytic oxidation (PEO) and plasma chemical anodization. Magnesium alloys are refined with an oxide ceramic layer which serves as wear and corrosion protection by way of anodic plasma chemical surface refinement. Surface refinement is an electrolytic method by which the workpiece is switched as an anode and the surface of the workpiece is converted into the corresponding oxides. Saline solutions are used as the electrolyte. The anodization takes place by way of plasma discharges in the electrolyte on the surface of the part that is to be coated. By the action of the oxygen plasma generated in the electrolyte on the metal surface, the metal is partially melted in a short time, and a firmly adhering oxide ceramic-metal bond is created on the workpiece.

Structurally, with an anodic plasma-chemical treatment, the developing passivation layer comprises a first thin layer, the so-called barrier layer, which is in direct contact with the metal substrate. On this barrier layer, a low-pore oxide ceramic layer is present, on which in turn a pore-rich oxide ceramic layer having about the same thickness is formed. The latter is particularly suited for impregnation with therapeutically active substances in pure form or appropriate galenics.

The electrolyte that is employed has a key function in the anodic plasma-chemical treatment. On the one hand, the ingredients of the electrolyte must be adjusted such that an ignition in the spirit of the method takes place at all. And on the other hand, the processes starting thereafter must be designed with respect to the purpose according to the invention. Surprisingly it was found that the presence of HF, which is an essential component in all conventional methods, results in a surface structure that is unsuitably excessively roughened and irregular for the special purposes of the surface refinement of biocorrodible implants made of magnesium alloys. This means that early and undesirable fragmentation can be observed, particularly in the case of mechanically stressed implants. Surprisingly it was found that foregoing HF, which is to say the use of a fluoride-free electrolyte, results in very consistent passivation layers if the electrolyte at the same time comprises ammonia, phosphoric acid, and boric acid.

A further, important method parameter of anodic plasma-chemical treatment is the applied voltage during the process. Thus, in the present example, it proved advantageous if the maximum end-point voltage is in the range of 100 to 400 volt, operating under direct current. The current densities preferably are 1 to 5 A/dm², such that layer formation speeds of approx. 1 μm/min are reached. Furthermore, the electrolyte temperature is preferably in the range of 30° C. to 50° C.

Under the above-described conditions, considerably finer sparks were observed than is the case in conventional applications of the anodic plasma-chemical method. This may also be an explanation for the more homogeneous layer structure. Due to the absence of the fluoride, the layer is less brittle because no MgF₂ can be formed. In addition, otherwise common pre-treatment of the material with hydrofluoric acid for activation was foregone, which surprisingly did not have any negative influence on the method.

The second method variant of the anodic treatment using an aqueous electrolyte, which at least comprises sodium permanganate and ammonium vanadate, also resulted in comparable structures. The layer thickness of the developing passivation layer was approx. 2 to 3 μm and proved to be well-bonded, but not brittle, so that flaking was not observed under mechanical stress.

Preferably a fraction of sodium permanganate is present in the electrolyte in the range of 1 to 5 g/l. A fraction of ammonium vanadate is preferably at 0.5 to 3 g/l. The treatment temperature is preferably in the range of 20° C. to 40° C. Preferably a direct current is applied in the range of 1 to 15 V.

Optionally, the electrolyte solutions comprise buffers, particularly alkaline buffers, such as EDTA, ethylene diamine, hexamethylene tetramine. Alkaline buffers support the formation of a barrier layer due to the high supply of OH⁻ ions thereof. In addition, they favorably affect the stability of the electrolytes.

The implant is made entirely, or at least in parts thereof, of the biocorrodible magnesium alloy. An alloy in the present case shall be understood as a metal structure, the main constituent of which is magnesium. The main constituent is the alloying constituent, the weight proportion of which in the alloy is the highest. A fraction of the main constituent is preferably more than 50 weight %, particularly more than 70 weight %.

The magnesium alloy is to be selected in the composition thereof such that it is biocorrodible. Biocorrodible as defined by the invention are alloys in which a degradation occurs in a physiological environment, which ultimately causes the entire implant, or the part of the implant composed of the material, to lose the mechanical integrity thereof. A possible test medium for testing the corrosion behavior of a potential alloy is synthetic plasma, as that which is required according to EN ISO 10993-15:2000 for biocorrosion analyses (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the alloy to be analyzed is stored in a closed sample container with a defined quantity of the test medium at 37° C. The samples are removed at time intervals—which are adapted to the corrosion behavior expected—ranging from a few hours to several months and analyzed for traces of corrosion in the known manner. The synthetic plasma according to EN ISO 10993-15:2000 corresponds to a blood-like medium and thereby is a possibility to reproducibly simulate a physiological environment as defined by the invention.

The term corrosion refers in the present example to the reaction of a metal material with the environment thereof, wherein a measurable change of the material is caused, which—when using the material in a component—results in an impairment of the function of the component. In the present example, a corrosion system comprises the corroding metal material and a liquid corrosion medium, which in the composition thereof simulates the conditions in a physiological environment, or is a physiological medium, particularly blood. With respect to the material, corrosion is influenced by factors such as the composition and pretreatment of the alloy, microscopic and sub-microscopic inhomogeneities, peripheral zone properties, temperature and mechanical stress states, and in particular the composition of a layer covering the surface. With respect to the medium, the corrosion process is influenced by the conductivity, temperature, temperature gradients, acidity, surface area to volume ratio, concentration difference, and flow velocity.

At the interphase region between the material and medium, redox reactions take place. For a protective and/or inhibiting effect, existing protective layers and/or the products of the redox reactions must form a sufficiently tight structure against the corrosion medium, have thermodynamic stability that is increased relative to the surrounding area, and be little soluble or insoluble in the corrosion medium. In the interphase region, or more precisely in a double layer forming in this region, adsorption and desorption processes take place. The processes in the double layer are shaped by the cathodic, anodic, and chemical sub-processes taking place there. In the case of magnesium alloys, generally gradual alkalinization of the double layer can be observed. Foreign matter deposits, contaminations, and corrosion products influence the corrosion process. The event during corrosion are therefore highly complex and cannot be predicted, or only to a limited extent, especially in connection with a physiological corrosion medium, which is to say blood or synthetic plasma, because comparative data is absent. Already for this reason, it is not part of the routine of a person skilled in the art to find a corrosion-inhibiting coating, which is to say a coating that is used only for a temporary reduction of the corrosion rate of a metal material of the composition described above in a physiological environment. This applies in particular to stents, which at the time of implantation locally are exposed to high plastic deformation forces. Conventional approaches having rigid corrosion-inhibiting layers are not suited for such requirements.

The corrosion process can be quantified by the provision of a corrosion rate. Swift degradation is associated with a high corrosion rate, and vice versa. Relative to the degradation of the entire shaped body, a surface that is modified as defined by the invention will result in a decrease of the corrosion rate. Over time, the corrosion-inhibiting coating according to the invention per se can be degraded, or it can protect the regions of the implant which it covers only to an every decreasing extent. Thus, the course of the corrosion rate for the entire implant is not linear. Rather, a relatively low corrosion rate occurs at the beginning of the onsetting corrosive processes, said rate increasing over the course of time. This behavior is considered a temporary decrease of the corrosion rate as defined by the invention and is a characteristic of the corrosion-inhibiting coating. In the case of coronary stents, the mechanical integrity of the structure should be maintained for a period of three months after implantation.

A further aspect of the invention relates to implants, which were obtained or are obtainable with the above-described methods.

Implants as defined by the invention are devices introduced into the body by a surgical procedure and comprise fastening elements for bones, such as screws, plates or nails, surgical suture material, intestinal clamps, vessel clips, prostheses in the area of hard and soft tissues, and anchoring elements for electrodes, particularly for pacemakers or defibrillators. The implant is made entirely of the biocorrodible material, or only in parts thereof. If only parts of the implant are made of the biocorrodible material, this part is to be coated accordingly.

The implant is preferably a stent. Stents of conventional build have a filigree structure made of metal struts, which is initially available in a non-expanded state for introduction into the body and is then widened into an expanded state at the site of the application. Stents have special requirements with respect to the corrosion-inhibiting layer. The mechanical stress of the material during the expansion of the implant (dilation) influences the course of the corrosion process, and it can be assumed that tension crack corrosion is intensified in the stressed regions. A corrosion-inhibiting layer should take this circumstance into consideration. Furthermore, a hard corrosion-inhibiting layer could flake during the expansion of the stent, and cracking in the layer is likely inevitable during expansion of the implant. Finally, the dimensions of the filigree metal structure must be observed, and if possible only a thin, yet uniform corrosion-inhibiting layer should be created. It was shown that the application of the coatings according to the invention fully, or at least largely, meets these requirements.

The corrosion-inhibiting coating obtainable by treatment with the conversion solution preferably has a layer thickness in the range of 1 μm to 10 μm.

The invention will be explained in more detail hereinafter based on exemplary embodiments and the associated illustrations. Shown are:

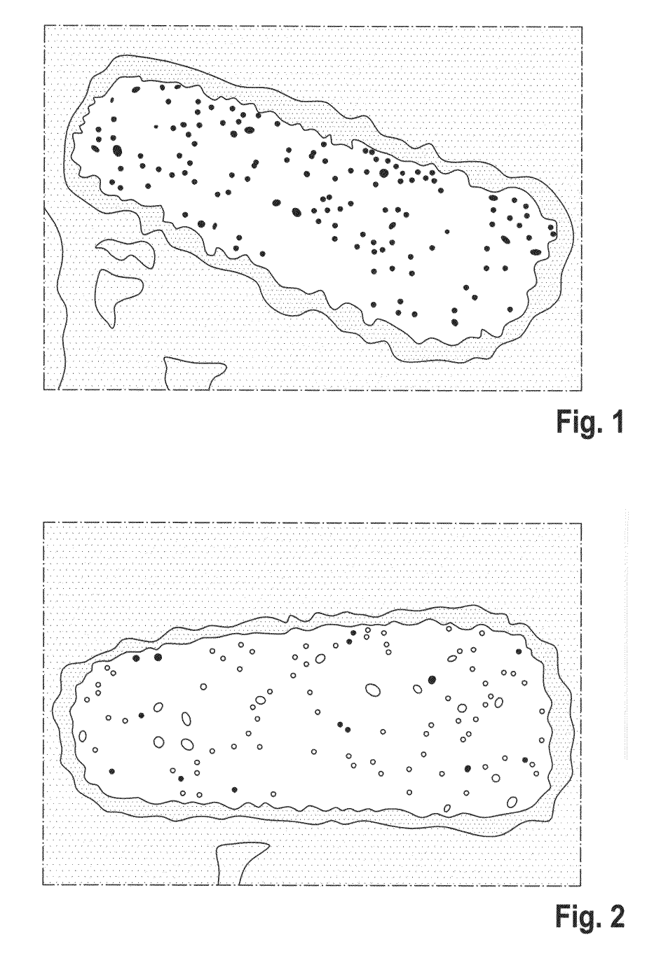

FIG. 1 Cross-section of a stent strut with anodic plasma-chemical treatment in the conventional manner,

FIG. 2 cross-section of a stent strut with anodic plasma-chemical treatment according to the invention.

### EXEMPLARY EMBODIMENT 1—ANODIC PLASMA-CHEMICAL OXIDATION

Stents made of the biocorrodible magnesium alloy WE43 (93 weight % magnesium, 4 weight % yttrium (W) and 3 weight % rare earth metals (E) except yttrium) were washed with isopropanol while applying ultrasound. After rinsing several times with deionized water, the stent was poled as an anode and immersed into an aqueous electrolyte solution having the following composition:
60 g/l H₃PO₄
35 g/l H₃BO₃
72.8 g/l NH₃
350 g/l Hexamethylene tetramine

Hexamethylene tetramine served the stabilization of an oxygen film on the anode and as pH buffer.

The parameters of the anodic oxidation were:

Current density 1.4 A/dm²

Bath temperature 40° C.

Max. voltage 340 V

After an exposure time of 10 minutes, the stent was rinsed with deionized water and then dried. The coating on the stents had a layer thickness of approximately 5 to 8 μm.

### COMPARATIVE EXAMPLE—ANODIC PLASMA-CHEMICAL OXIDATION IN THE PRESENCE OF HF

For comparison, stents were treated under the same conditions as outlined in exemplary embodiment 1, however with the difference that the electrolyte contained 30 g/l hydrofluoric acid.

FIG. 1 shows a cross-section of a stent strut, which was surface-treated according to the comparative example, while FIG. 2 shows a cross-section of a strut treated according to exemplary embodiment 1 of the invention. As is apparent, the created passivation layer is significantly more uniform with the method according to the invention, which produces the above-described advantages when the structure experiences mechanical stress.

### EXEMPLARY EMBODIMENT 2—ANODIC OXIDATION

Stents made of the biocorrodible magnesium alloy WE43 (93 weight % magnesium, 4 weight % yttrium (W) and 3 weight % rare earth metals (E) except yttrium) were washed with isopropanol while applying ultrasound. After rinsing several times with deionized water, the stent was immersed in the wet state for 5 minutes into an aqueous electrolyte solution heated to 30° C. and comprising 2.7 g/l NaMnO₄ and 1 g/l NH₄VO₃. Furthermore, a direct current of 5 V was applied to intensity the passivation effect and the stent was poled as an anode. After removing the stent out of the electrolyte solution, the implant was rinsed several times with deionized water and thereafter dried for 30 minutes in a recirculating dryer at 120° C.

1. A method for producing a corrosion-inhibiting coating on an implant made of a biocorrodible magnesium alloy, comprising the following steps:
(i) Providing the implant made of a biocorrodible magnesium alloy; and
(ii) anodic plasma-chemical treatment of the implant surface in an aqueous, fluoride-free electrolyte, which comprises at least ammonia (NH₃), phosphoric acid (H₃PO₄) and boric acid (H₃BO₃).

2. A method for producing a corrosion-inhibiting coating on an implant made of a biocorrodible magnesium alloy, comprising the following steps:
(i) Providing the implant made of a biocorrodible magnesium alloy; and
(ii) anodic treatment of the implant surface with an aqueous electrolyte, which comprises at least sodium permanganate (NaMnO₄) and ammonium vanadate (NH₄VO₃).

3. An implant having a corrosion-inhibiting coating, obtained or obtainable by a method according to claim 1.

4. An implant having a corrosion-inhibiting coating, obtained or obtainable by a method according to claim 2.

5. The implant according to claim 3, wherein the implant is a stent.

6. The implant according to claim 4, wherein the implant is a stent

7. The implant according to claim 3, wherein the corrosion-inhibiting coating has a layer thickness in the range of 1 μm to 10 μm.

8. The implant according to claim 4, wherein the corrosion-inhibiting coating has a layer thickness in the range of 1 μm to 10 μm.

9. An implant according to claim 3, wherein the corrosion-inhibiting coating is loaded with a pharmaceutical agent at least in some regions.

10. An implant according to claim 4, wherein the corrosion-inhibiting coating is loaded with a pharmaceutical agent at least in some regions.

11. A method for applying a corrosion-inhibiting coating to a stent made of a biocorrodible magnesium alloy and having a surface, the method comprising the steps of:
applying an anodic plasma-chemical treatment to the stent surface in an aqueous, fluoride-free electrolyte that comprises at least ammonia (NH₃), phosphoric acid (H₃PO₄) and boric acid (H₃BO₃) to result in a corrosion inhibiting coating having a thickness of between about 1 μm to 10 μm; and,
loading at least some regions of the coating with a pharmaceutical agent.
